(19) United States
(12) Patent Application Publication
(10) Pub. No.: US 2014/0007900 A1
(43) Pub. Date: Jan. 9, 2014

(54) METHOD OF REMOVING DENTAL CEMENT FROM DENTAL RESTORATIONS

(71) Applicant: Joseph Fish, Tel Aviv (IL)

(72) Inventor: Joseph Fish, Tel Aviv (IL)

(21) Appl. No.: 13/984,375

(22) PCT Filed: Feb. 20, 2012
(86) PCT No.: PCT/IL12/00085

§ 371 Date: Aug. 13, 2013

(30) Foreign Application Priority Data
Feb. 21, 2011 (IL) 211327
Nov. 10, 2011 (IL) 216285

(51) Int. Cl. B08B 3/08

(52) U.S. Cl. CPC B08B 3/08
USPC 134/1; 134/6; 134/32; 134/34; 510/161

(57) ABSTRACT

The present invention provides a liquid composition for removing dental cement from the surface of non-living objects, comprising i) an inorganic base; ii) a complexing agent; iii) optionally a surfactant; and iv) an aqueous solvent. In a preferred embodiment, the composition comprises a surfactant, and an aqueous solvent comprising water mixed with a polar solvent miscible with water, preferably alcohol. Said objects to be cleaned comprise workpieces and instruments used in restoration work. Said workpieces may comprise crowns, bridges, inlays, and other prostheses. Said instruments may, comprise, for example, tools used during the dental operation which contacted the cement.

### FIELD OF THE INVENTION

The present invention relates to the field of restorative dentistry, and more particularly it relates to cleaning dental workpieces or instruments, intended for temporary or repeated use, from dental cements.

### BACKGROUND OF THE INVENTION

The dental cement is a shapeable material filling the space between two surfaces, increasing their friction coefficient and restricting their mutual movement. The surfaces typically comprise temporary or final dental prosthesis. The cements may also act as cavity-lining materials, pulp-protecting agents, luting agents, and auxiliary agents for other purposes. Some dental works require temporary cements, others permanent cements.

In many uses, the dental cement should form a strong connection with the contacted surfaces or it should provide a tight sealing. The cement hardens through various chemical reactions, and firmly attaches to the surfaces it contacts. The items comprising said surfaces include, for example, dental crowns and bridges. Dental cements may be prepared before the use by admixing a powder into a liquid, or by mixing two paste-like components, while forming a paste that must be applied before getting hard. The cements may, for example, comprise metal oxides or silicates, and acids like phosphoric acid or carboxylic acids. Typical dental-cement composition comprises at least one component of the following three groups: metal oxide or hydroxide, polyacrylates, and derivatives of phenol or benzoic acid; mostly, at least two components are included. Said metal oxide or hydroxide is often zinc oxide, and said derivative of phenol or benzoic acid is often eugenol. Very frequently used is a cement comprising zinc oxide and eugenol. Eugenol has antiseptic effects, and when mixed with zinc oxide, it makes good dental restorative agent. Zinc oxide-eugenol compositions may be reinforced with polymer, and are used, for example, as a non-irritating final cement for finished crowns, bridges, and inlays.

Dental cements exhibit considerable stability; for example, zinc carboxylate cements were shown to be stable up to 17 years [Leloup J. M. et al.: J. Mat. Sci.: Materials in Medicines 9 (1998) 493-6]. On one hand, the use of cements requires strength and stability; on the other hand, such bonding strength makes it difficult to remove cement residues from restoration workpieces between consequent sessions of the patients, or from instruments and other objects to be reused. Two variants of chemical treatment for disintegration of dental cement are known in the art: a) treatment with acid media, and b) treatment with alkaline media. In the category of acid treatments, the use of aqueous solutions of organic acids is common. For example, U.S. Pat. No. 4,162,172 discloses a method of removing a hardened dental cement from a surface by applying solution of citric acid. In a preferred practice of this invention 200 grams of citric acid is added to 500 ml of warm tap water and the solution is placed in an ultrasonic cleaner. The citric acid may be anhydrous or monohydrate. Although the monohydrate has the disadvantage of being more expensive, both forms work equally well as long as the 200 grams monohydrate citric acid equals the 200 grams of anhydrous citric acid. The dental product is then immersed in the cleaner and the carboxylate or zinc oxyphosphate cement is quickly removed by the combination of actions from the citric acid and from the ultrasonic cleaner. Disadvantage of this method consists in an excessive consumption of citric acid (solutions with concentrations higher than 35%) that made the treatment to be expensive.

U.S. Pat. No. 4,806,173 relates to cleansing compositions and methods of their application. In particular, it relates to a method for cleansing dental appliances, such as artificial dentures. The cleansing composition consists essentially of an aqueous solution of one carboxylic acid, such as acetic, succinic, maleic, citric, mandelic, lactic, fumaric, or derivatives thereof such as anhydrides, hydroxy acids, halogenated, acids and aralkyl acids. The dental appliance is soaked in the aqueous solution overnight, and washed with water. Depending on the amount of the tartar deposits, a 3-10% concentration of the acid in water is used. A disadvantage of this method is a long time necessary for cleaning, several hours, necessitating at least two visits of the patient in the clinic.

U.S. Pat. No. 5,980,641 presents a method for cleaning dentures in which a solution of about 5% carboxylic acid, an artificial sweetener, and a flavoring is prepared, the dentures are placed therein and agitated. After agitation, the dentures are removed from the solution and are insertable into a wearer's mouth immediately without being rinsed, brushed or soaked. The solution is prepared by placing an amount of 25% a carboxylic acid solution to provide 5% concentration, possibly using an ultrasonic cleaning apparatus. A disadvantage of this method consists in an excessive consumption of citric acid (solutions with concentrations higher than 20%), making the treatment expensive.

U.S. Pat. No. 5,154,613 describes removing the dental cement in a dissolving bath which contains a metal hydroxide in a concentration of between 0.5 and 3.0 molar, and a chelating agent in a concentration between 0.25 and 2.0 molar. The purpose of the metal hydroxide is to chemically breakdown the metal oxide-polycarboxylic acid matrix (strong) by hydrolyzing the metal-acid bonds in this matrix. The function of the chelating agent is to chemically bond with the metal hydroxide in the cement, and to breakdown the matrix (weak) formed between the cement metal hydroxide and polycarboxylic acid. The preferred chelating agent is sodium EDTA in a concentration of from 0.50 to 1.25 molar. Disadvantages of this method consist in using concentrated basic media which are difficult to safely handle, and the high consumption of chelating agents. US 2008/0096784 relates to a composition for cleaning dental instruments comprising (a) one or more amino acids and/or salts thereof (aminocarboxylates) in a total amount from 20 to 70 parts by weight, (b) one or more alpha-hydroxy acids and/or salts thereof in a total amount from 15 to 45 parts by weight, (c) one or more alkali metal carbonates and/or bicarbonates in a total amount from 0 to 50 parts by weight, (d) one or more surfactants in a total amount from 0 to 5 parts by weight, (e) one or more tabletting auxiliaries in a total amount from 0 to 10 parts by weight, one or more corrosion inhibitors in a total amount from 0 to 5 parts by weight, and (g) one or more other additives in a total amount from 0 to 55 parts by weight, wherein components (a), (b), (c), (d), (e), (f) and (g) are present in total in an amount of 100 parts by weight, and wherein the components are chosen such that the pH of a solution prepared by mixing the composition with 4,000 parts by weight of water is in the range from 7.0 to 9.5. Disadvantage of this method is that the time necessary for cleaning is several hours, possibly more than 8 hours, which necessitates more visits of the patient at the dentist's clinic.

A continual need is felt for additional cleaning compositions, particularly cleaning compositions for removing temporary cements. It is therefore an object of the invention to provide a new composition for removing dental cements from dental workpieces, thereby facilitating to dentists the use of the dental cements, particularly temporary cements.

It is another object of this invention to provide a composition for removing a variety of dental cements from dental instruments and equipment intended for repeated use.

It is also an object of this invention to provide a composition for removing a temporary dental cement from dental prosthesis, such as inlays, crowns, and bridges.

It is still another object of this invention to provide a composition for cleaning dental tools to which said tools are resistant.

It is further an object of this invention to provide a method of removing residual dental cements off the surface of dental instruments without damaging their surface and enabling their repeated use.

Other objects and advantages of present invention will appear as description proceeds.

### SUMMARY OF THE INVENTION

The present invention provides a liquid composition for removing dental cement from the surface of non-living objects, comprising i) an inorganic base; ii) a complexing agent; iii) optionally a surfactant; and iv) an aqueous solvent. In a preferred embodiment, the composition comprises a surfactant, and an aqueous solvent comprising water mixed with a polar solvent miscible with water, preferably alcohol. Said objects to be cleaned comprise workpieces and instruments used in restoration work. Said workpieces may comprise crowns, bridges, inlays, and other prostheses. Said instruments may comprise, for example, tools used during the dental operation which contacted the cement. Said inorganic base may be selected from hydroxides and carbonates, examples being KOH, NaOH, LiOH, NH₄OH, sodium carbonate, potassium carbonate, sodium bicarbonate, and potassium bicarbonate. Said complexing agent is preferably selected from aliphatic amines, aliphatic hydroxylamines, and mixtures thereof.

In one embodiment, the complexing agent comprises a mixture of one or more aliphatic amine and/or aliphatic hydroxylamine. The amount of the amine included in the solutions of the present invention may vary from about 0.5 to about 30 g/l. Examples of the amines which are useful include ethylenediamine, diaminopropane, diaminobutane, N,N,N,N-tetramethyldiaminomethane, diethylenetriamine, 3,3-aminobispropylamine, triethylene tetramine, monoethanolamine, diethanolamine, triethylanolamine, N-methyl hydroxylamine, 3-amino-1-propanol, N-methyl ethanolamine, etc.

Said surfactant may be selected from nonionic surfactants witch high dipole moments in aqueous solution and are highly hydrated. Examples of nonionic surfactants are alkylene oxide adducts, such as those obtained by addition of ethylene oxide and/or propylene oxide onto fatty alcohols, fatty acids, fatty to acid glycerides, phenols, fatty amines, and alkylphenols, it also being possible for the terminal hydroxyl groups of these polyglycol ether derivatives to be etherified, esterified or acetylated.

The aqueous solutions of the present invention may be prepared by dissolving the various components mentioned above in water. The components may be mixed with water in any order. In one aspect of the invention, water constitutes at least 80 wt % of the composition. In a preferred embodiment water constitutes 85 wt % of the solvent or more. In one preferred embodiment of the invention, a cleaning composition comprises an alkali metal base, a complexing agent such as amine, a nonionic surfactant and water. An example of a preferred composition for removing cements from the dental items is a mixture comprising an alkali metal base, monoethanolamine, polyethylene oxide, and water. In another aspect of the invention, the liquid solution for cleaning the dental items comprises a water/alcohol mixture, preferably water/ethanol mixture, wherein water constitutes from 10 to 100 wt % of said aqueous solvent.

The invention provides a method of removing dental cement from the surface of non-living objects, comprising i) immersing said object in a composition comprising at least one inorganic base, at least one complexing agent, preferably at least one surfactant, and water for a time period allowing at least partially to dissolve the cement and reduce its attachment to said surface; and ii) exposing said object to mechanical treatment. Said time period may be typically 10 minutes or less than 30 minutes. The time period may be shorter. In case of need, longer treatment is used. Said mechanical treatment comprises, for example, one or more activities selected from the group consisting of washing with a water jet, scraping, rubbing, subjecting to mechanical vibrations, and subjecting to ultrasound. Said step of subjecting the cleaned object to ultrasound preferably comprises placing said object in a bath sonicator while immersed in a composition as described above.

### DETAILED DESCRIPTION OF THE INVENTION

It has now been found that a composition comprising an inorganic base, a complexing agent, a surfactant, and water removes a temporary dental cement surprisingly well from the surface of crowns and bridges, and other temporary or final prostheses. For example, potassium hydroxide in ethanol, with an admixture of water, with an amine and a surfactant dissolved in said water, released the hardened zinc oxide-based cement within ten minutes of immersion. Moreover, the same composition, during the same time, enabled to efficiently clean also dental instruments.

Said inorganic base may be selected form hydroxides and carbonates. Examples of inorganic bases include KOH, NaOH, LiOH NH₄OH, sodium bicarbonate, potassium bicarbonate. Preferably, said inorganic base is selected from alkali hydroxides.

Generally, complexing agents are materials capable of donating at least one electron pair, thereby forming a coordination complex. The agents may include ethers, water, and amines, examples including crown ethers, dioxane, aliphatic amine, aliphatic hydroxylamine. In the preferred embodiments of the invention, complexing agents include compounds comprising nitrogen or oxygen providing said electron pair, like for example aliphatic amine, an aliphatic hydroxylamine, or mixtures thereof. Said amines may include dimethylamine, diethylamine, EDTA, etc. The amount of the amine included in the solutions of the present invention may vary from about 1 to about 50 g/l. Other examples of the amines which are useful include ethylenediamine, diaminopropane, diaminobutane, N,N,N,N-tetramethyldiaminomethane, diethylenetriamine, 3,3- aminobispropylamine, triethylene tetramine, monoethanolamine, diethanolamine, triethylanolamine, N-methyl hydroxylamine, 3-amino-1-propanol, N-methyl ethanolamine, etc. Preferably, said complexing agent comprises aliphatic hydroxylamines.

The compositions according of the present invention can comprise one or more surfactants. The surfactants can deposit themselves between the various phases of the system to be cleaned and reduce the surface tension of the water at the dental cement residue interface. Said surfactant may be selected from nonionic surfactants witch high dipole moments in aqueous solution and are highly hydrated. Examples of nonionic surfactants are alkylene oxide adducts, such as can be obtained by addition of ethylene oxide and/or propylene oxide onto fatty alcohols, fatty acids, fatty acid glycerides, phenols, fatty amines and alkylphenols, it also being possible for the terminal hydroxyl groups of these polyglycol ether derivatives to be etherified, esterified or acetalized. Preferably, said surfactant is selected from C₁₆-C₁₈ fatty alcohol ethoxylates

In one aspect of the invention, a liquid composition for removing dental cement from the surface of non-living objects comprises inorganic base, complexing agent, and/or an agent reducing the surface tension of water. In one embodiment, such a composition comprises water, inorganic base, and an admixture of lower aliphatic alcohol as an agent for reducing the water surface tension. In another embodiment, such a composition comprises inorganic base in alcohol, and an admixture of water as a complexing agent. In a still other embodiment, the composition comprises alcohol/water mixture and an inorganic base. In a further embodiment, the composition comprises ethanol/water mixture, an inorganic base, and optionally a surfactant and/or an amine. In an important aspect of the invention, a liquid composition for removing dental cement from the dental items comprises an aqueous solvent, an inorganic base, and an amine or a surfactant or both, wherein the solvent is a mixture water/alcohol, preferably comprising from 5 wt % to 95 wt % water; an example being a mixture comprising water/ethanol mixture containing from 10 wt % to 90 wt % water, an alkali metal hydroxide, and optionally an aliphatic amine and/or a nonionic surfactant. In one aspect, the composition of the invention comprises an aqueous solvent, comprising water and a hydrophilic solvent miscible with water, such as dimethylformamide, formamide, acetone, dimethylsulfoxide, alcohols, dioxane, and acetonitrile.

Preferred solvents include lower alcohols. When considering health issues, safety, and comfortable work, ethanol is one of preferred solvents. In one aspect of the invention, water constitutes at least 80 wt % of the solvent, or at least 85 wt %, or at least 90 wt %, or at least 95 wt %, examples comprising 85 wt % or 95 wt % or 99 wt %. In other aspect, the solvent may constitute between 75 wt % and 95 wt % of the composition, examples comprising about 75 wt %, about 80 wt %, and about 90 wt %. The inorganic base may comprise KOH in an amount of from 7 to 15 wt %, such as between 10 and 12 wt %. The complexing agent may be amine, for example from 0.5 to 1.5 wt % ethylamine. The surfactant may be, for example, a non-ionic surfactant in an amount of from 0.05 to 0.15 wt %, such as stearyl ethoxylate.

Dental works like restoration and abutment require good cements, and said good cements later require the means for efficient removal of the cement residues. Various temporary dental cements are used in cementation of temporary or final dental products, such as crowns, bridges; when the dentist removes a temporarily cemented workpiece, it is essential to clean it before re-attaching from adhering remnants of dental cement. This is often done by mechanical means, while employing repeated scraping and rinsing with water; this is time-consuming, frequently insufficient, and often causing mechanical damages to the treated items, or even wounds to the personnel. In many cases, the cements comprise zinc oxide, either with eugenol or with other additives. The invention provides a variety of new cleaning compositions, essentially liquid compositions based on a hydrophilic solvent such as water or an organic solvent miscible with water, preferably alcohol or alcohol/water mixtures, enabling to select a suitable composition for many different situations. For example, enabled is a citric acid-free composition.

The invention thus provides the means for restoration of teeth, mainly in the stage of applying temporary or final dental prosthesis, such as crowns, bridges, inlays, and onlays, which items can be efficiently cleaned with a composition according to the invention before reuse. The invention further facilitates using temporary cements in dental restoration work, enabling to efficiently and safely clean dental workpieces like prostheses, as well as instruments, equipment and products contacting the cement during the dental operation.

A suitable cleaning composition should not attack the materials of which the cleaned instruments are made, such as ceramic, plastics, porcelain, precious metals, stainless steel, various metal alloys, etc. In accordance with the invention, the cleaned item is immersed in a liquid composition according of the invention and further mechanically treated, wherein the mechanical treatment comprises one or more of washing with a water jet, rubbing, subjecting to mechanical vibrations, subjecting to ultrasound. Typically, after immersing an instrument in a liquid composition of the invention, the cement is at least partially dissolved, and the adherence of the cement to the cleaned item is reduced; the cement, even if not dissolved, is easily removed off the item surface. For example, after immersing the item from about five minutes to thirty minutes, the weakened cement is flushed away with a stream of water.

Temporary cements to be released from the surface of dental items according to the invention may include cements based on zinc oxide, either with or without eugenol. The cements may be reinforced with polymeric components. In a preferred embodiment of the invention, a cement comprising metal oxide is removed from the dental workpieces, instruments, and other equipment, said oxide being often zinc oxide. Said workpieces are typically dental prostheses, final or temporary.

The items or objects being cleaned from the dental cements preferably include devices repeatedly used in dental restorations, including workpieces for making prostheses, and instruments used during the operations. The simple instruments may include tools like spatulas or vessels. The surface of said items or object is efficiently rid of the cement residues without damaging the item or object for repeated use. A skilled person will understand that the compositions of the invention should never contact the living body.

A preferred composition of the invention comprises potassium or sodium hydroxide, water, optionally ethanol, amine, and surfactant; for example, 8-12 grams of KOH, 0.8-1.2 grams of monoethanolamine, and 0.02-0.08 grams C₁₇ fatty alcohol ethoxylate in 100 ml water.

The invention will be further described and illustrated in the following example.

### EXAMPLES

### Example 1

A solution according to the invention, comprising 12 g potassium hydroxide, 20 ml water, and 100 ml ethanol was tested with the following constructions materials used for dental workpieces (some of the names comprise Trade Marks or Trade Names): Duralay, Snap, Biofil, Silmat, Temron C, composite used crowns, Plasket, Option, Rexilium, gold, porcelain crown, Aristaloy, and JetAcryl. After the immersion of 10 minutes or 30 minutes or 12 hours, the weight was determined. The results showed that the composition does not attack common materials used for dental items. Items with hardened cement were immersed for 10 minutes and flushed with water, thereby removing the cement residues. Of course, a skilled person will check the alkalinity on the surface of the treated item before its reusing.

### Example 2

A solution according to one of preferred embodiments of the invention, comprising 10 grams of KOH, 1 gram of monoethanolamine, 0.06 grams C₁₇ fatty alcohol ethoxylate in 100 ml water was tested with the following constructions materials used for dental workpieces (some of the names comprise Trade Marks or Trade Names): Duralay, Snap, Biofil, Silmat, Temron C, composite used crowns, Plasket, Option, Rexilium, gold, porcelain crown, Aristaloy, and JetAcryl. After the immersion of 10 minutes or 30 minutes or 12 hours, the weight was determined. The results showed that the composition removed the cement within 10 minutes but did not attack common materials used for dental items. Items with hardened cement were immersed for 10 minutes and flushed with water, thereby removing the cement residues. Of course, a skilled person will check the alkalinity on the surface of the treated item before its reusing.

While this invention has been described in terms of some specific examples, many modifications and variations are possible. It is therefore understood that within the scope of the appended claims, the invention may be realized otherwise than as specifically described.

1. A composition for removing dental cement from the surface of non-living objects, comprising
i) an inorganic base;
ii) a complexing agent;
iii) a surfactant; and
iv) aqueous solvent.

2. A composition according to claim 1, wherein said objects comprise workpieces and instruments used in restoration work.

3. A composition according to claim 2, wherein said workpieces comprise crowns, bridges, inlays, and other prostheses.

4. A composition according to claim 1, wherein said inorganic base is selected from NaOH, KOH, and LiOH.

5. A composition according to claim 1, wherein said complexing agent is selected from aliphatic amines, aliphatic hydroxylamines, or mixtures thereof.

6. A composition according to claim 1, wherein said surfactant is selected from nonionic surfactants.

7. A composition according to claim 1, in which said aqueous solvent comprises at least 80 wt % of said composition.

8. A composition according to claim 1, wherein said aqueous solvent comprises from 10 to 100 wt % water.

9. A composition according to claim 1, comprising an alkali metal base, an amine, a nonionic surfactant, and water.

10. A method of removing dental cement from the surface of non-living objects, comprising
i) immersing said object in a composition according to claim 1 for a time period allowing to at least partially dissolve the cement and to reduce the attachment of said cement to said surface; and
ii) exposing said object to mechanical treatment.

11. A method according to claim 10, wherein said mechanical treatment comprises at least one activity selected from the group consisting of washing with a water jet, scraping, rubbing, subjecting to mechanical vibrations, and subjecting to ultrasound.

12. A method according to claim 11, wherein said subjecting to ultrasound comprises placing said object in a sonicator bath.
